(19) United States
(12) Patent Application Publication
(10) Pub. No.: US 2004/0241123 A1
(43) Pub. Date: Dec. 2, 2004

(54) Suspension free and elastomer free antiperspirant cream

(76) Inventors: Christine Popoff, Morganville, NJ (US); Marian Holerca, Somerset, NJ (US)

Correspondence Address: Colgate-Palmolive Company, 909 River Road, P.O. Box 1343, Piscataway, NJ, 08855-1343, US

(21) Appl. No.: 10/449,289

(22) Filed: May 30, 2003

(51) Int. Cl. A61K 7/32; A61K 7/06; A61K 7/11

(52) U.S. Cl. 424/70.12

(57) ABSTRACT

The invention is a suspension free and elastomer-free composition comprising: (a) 0.1-30 weight % of an antiperspirant active having a low metal to chloride ratio in the range of 0.9 to 1.5:1; (b) 7-28.4 weight % of one or more volatile silicones having a flash point of 100 degrees C. or less; (c) 0.6-2.0 weight % of a silicone surfactant having an HLB value≦8; (d) 30-70 weight % water; (e) 0-3 weight % of a water soluble glycol or polyglycol; (f) 1-5% silicone emollient; and (g) 0-3 weight % of a non-siliconized organic fragrance solubilizer; wherein the composition is optically a white suspension-free cream and has a viscosity greater than 150,000 centipoise and a ratio of oil phase to water phase in the range of 10:90 to 30:70.

### FIELD OF THE INVENTION

The present invention is directed to a suspension-free white cream composition which is a high viscosity (>150,000 cps at 21 degrees C.), water in oil emulsion, free of elastomer, soap and borate gelling agents as well as any surfactants having an HLB>9. The compositions provide enhanced skin feel and has at least equal antiperspirant and/or deodorant efficacy as a clear water-in-oil emulsion gel. The suspension free cream is comprised of a glycine-complexed antiperspirant active (preferably with a lower metal to chloride ratio) along with a monovalent or divalent ionizable salt and a reduced amount of glycols in the active phase as well as low levels of nonvolatile ingredients in the oil phase. This cream is formulated as refractive white products having reduced whitening and tack as well as reduced skin irritation.

### BACKGROUND OF THE INVENTION

Antiperspirant products are well known in the art. Antiperspirants have appeared in the marketplace in varied dosage forms, such as sticks, soft solids, soft gels, roll-on, aerosols and creams. Generally, these dosage forms include a solution of the active ingredient in a suitable solvent, a suspension of the active ingredient in a non-solvent, or a multiphasic dispersion or emulsion in which a solution of the active ingredient is dispersed in some continuous phase or in which the solubilized active ingredient constitutes a continuous phase.

Of the above-referred-to dosage forms, the stick form is an example of a solid form, and the soft solid and soft gel are thickened forms which may or may not be solid (for example, under some circumstances, gels can flow). The stick form can be distinguished from a soft solid or soft gel in that, in a stick, the formulated product can retain its shape for extended time periods outside the package, the product not losing its shape significantly (allowing for some shrinkage due to solvent evaporation). Adjustment of amounts of gelling or thickening agents can be used in order to form a soft gel or stick.

Soft gels or soft solids can be suitably packaged in containers which have the appearance of a stick, but which dispense through apertures (for example, slots or pores) on the top surface of the package. The soft solid products have also been called soft sticks or “smooth-ons”, and hereinafter are generically called “soft solids”. Reference is made to U.S. Pat. No. 5,102,656 to Kasat, U.S. Pat. No. 5,069,897 to Orr, and U.S. Pat. No. 4,937,069 to Shin, each of which discloses such soft solids, including physical characteristics thereof such as viscosity and hardness. The contents of each of these three U.S. patents are incorporated herein by reference in their entirety for description of characteristics of soft solids and suitable packaging for such products.

Recently there has been significant activity in developing antiperspirant cream compositions. U.S. Pat. Nos. 5,902,570, 5,891,424, 5,891,425 to Procter and Gamble describe anhydrous antiperspirant cream compositions containing particulate antiperspirant actives.

U.S. Pat. No. 5,972,319 assigned to the same owner as this case describes a stick composition comprised of a gelling agent consisting of high melting point waxes, a solvent for the gelling agent, a non-volatile emollient which is not a silicone with a refractive index>1.4460, an effective amount of antiperspirant and/or deodorant, and an ethoxylated fatty acid surfactant.

U.S. Pat. No. 6,267,970 assigned to Avon describes an anhydrous cream having a wax, powder component and volatile component.

U.S. Pat. No. 6,492,459 assigned to Wacker describes an aqueous firm cream comprised of one or more organosilicone compounds, one or more emulsifiers, one or more organic solvents immiscible with water.

U.S. Pat. No. 5,939,056 assigned to Helene Curtis describes an anhydrous underarm cream composition suitable for topical application to the human skin comprising of an antiperspirant and/or deodorant agent, a carrier, a silica structurant and an alkyl methicone wax having the general formula:

(CH₃)₃Si—[O—Si(R)(CH₃)]ₓ—O—Si(CH₃)₃.

U.S. Pat. No. 4,948,578 assigned to Unilever describes an antiperspirant composition in the form of a transparent stick which is an oil-in-water emulsion comprising antiperspirant salts, 10-40% of nonionic surfactant and 5-50% of a liquid oil immiscible with water.

U.S. Pat. No. 4,122,029 assigned to Dow Corning describes emulsions of 1-70 weight percent of a polar liquid in a non-polar base liquid (17-97.5 weight %) which are prepared with 0.5-3 weight % of a water-in-oil surfactant having an HLB value from 2-10 and 1-10 weight polydiorganosiloxanepolyoxyalkylene copolymers.

U.S. Pat. No. 4,268,499 also assigned Dow Corning describes a water in oil antiperspirant emulsion composition consisting essentially of 30-60 parts by weight of an aqueous solution of an astringent, 27-67.5 parts by weight of a volatile liquid, 0.5-3 parts by weight of an organic water-in-oil surfactant, 1 to 5 parts by weight of polydiorganosiloxanepolyoxyalkylene copolymer and 1 to 5 parts by weight of an organic oil-in-water surfactant.

U.S. Pat. No. 4,719,103 assigned to American Cyanamid Company describes a water-in-oil antiperspirant composition which is comprised of 1-3 weight % of a non-ionic surfactant having a HLB value in the range of 6 to 9 and includes 10 to 30 parts by weight of at least one solid alkanol containing at least 12 carbon atoms.

U.S. Pat. No. 4,725,431 also assigned to American Cyanamid Company describes a water-in-oil antiperspirant composition which comprises emulsifying the aqueous phase in the oil phase in the presence of about 1 to 3 weight percent of at least one C₁₂ to C₂₀ saturated fatty acid ester of polyglycerol containing about 2 to 10 repeating glycerol units.

PCT Publication WO 97/48373 assigned to Unilever describes an antiperspirant or deodorant cosmetic composition suitable for topical application to the human skin, comprising: i) an antiperspirant or deodorant active; ii) a moisturizing cream; and, optionally, iii) a carrier for the antiperspirant or deodorant active.

There has been significant activity in developing clear antiperspirant sticks and soft gels, particularly to provide sticks and soft gels having increased efficacy (for example, by providing increased amounts of the antiperspirant active in the sticks and soft gels), improved cosmetic characteristics (including reduced whitening, reduced residue and reduced tack), and reduced skin irritation potential (e.g., providing a product that is “mild”).

U.S. Pat. No. 4,944,938 to Potini discloses clear, non-alcoholic, quick drying, antiperspirant and deodorant gels, which are stable both at room temperatures and at higher temperatures, are non-stinging and leave no white residue on the skin, the gel is free of gelling agents, waxes, clays, or monohydric alcohols having 2-8 carbon atoms. The gels use 3-5 carbon atom trihydric alcohols as coupling agents, which act as solublizers in the system and keep the system stable and clear. The gels can include an aluminum active salt; a volatile water-insoluble emollient, such as isostearyl benzoate: a soluble emollient such as cetyl ether; solubilizers such as propylene glycol and glycerin; volatile siloxanes; and water.

Some cellulosic materials, such as hydroxypropylcellulose, among others, are compatible with polyvalent metal salts and have been used in the manufacture of clear lotions. These cellulosic materials, however, must be prepared with a high percentage of water or alcohol in order to insure solubilization of the active ingredient. The resulting formulations, in addition to a high irritation potential, are tacky and low in efficacy, when alcohol-based; and exhibit tackiness and along drying time when water-based.

Clear antiperspirant soft gels (which have been dispensed from containers having the appearance of stick) have recently been marketed, consisting of viscous, high-internal-phase emulsions. These soft gels exhibit some advantages over the aforementioned sticks, particularly acetal-based clear sticks, in that the selection of formulation ingredients is less restricted (for example, water can be used), and often tack can be reduced significantly. Concerning these emulsions, note U.S. Pat. No. 4,673,570 to Soldati and U.S. Pat. No. 4,900,542 to Parrotta, et al. These two U.S. patents disclose clear gelled antiperspirant compositions free of waxes and conventional gelling agents, containing a volatile silicone fluid, a silicone emulsifier, a destabilizing auxiliary emulsifier, water, non-volatile emollient, a coupling agent, an active antiperspirant component and ancillary agents such as perfume, coloring agents, etc. The silicone emulsifiers a cyclomethicone-dimethicone copolyol silicone fluid marketed by Dow Corning Corporation under the trademark DOW CORNING 3225C formulation. In particular, U.S. Pat. No. 4,673,570 claim 1 is “from about 10-25% by weight of a cyclomethicone-dimethicone copolysilicone fluid (same as 1.0-2.5% on an actives basis). The contents of these two U.S. patents are incorporated herein by reference in their entirety with respect to the features of the inventions described therein.

Also to be noted is PCT (International application) Publication No. WO 92/05767. This patent document discloses a clear gel-type cosmetic product having a viscosity of at least about 50,000 cps at 21 degrees C. and a refractive index of 1.3975-1.4025 at 21 degrees C., and having an optical clarity better than 50 NTU (Nephelometric Turbidity Units) at 21 degrees C., the product being an emulsion with a water phase having an active ingredient incorporated therein and with an oil phase. The refractive indices (measured at 5893 Angstroms) of the water and oil phases match to within 0.0004. The oil phase includes an emulsifier which when properly mixed with the water phase component yields a water-in-oil emulsion, and the water phase includes one or a combination of various polar species such as water, propylene glycol, sorbitol and ethanol. The water phase includes the deodorant and/or antiperspirant active ingredient. The contents of this PCT (International application) Publication No. 92/05767 are incorporated herein by reference in their entirety.

U.S. Pat. No. 6,007,799, assigned to the same owner as this case, describes clear cosmetic gels that are water-in-oil emulsions and which comprise at least one coupling agent, silicone fluids and an alkoxylated, alkyl substituted silicone surface active agent.

U.S. Pat. Nos. 5,587,153 and 5,863,525 issued to Gillette also describe gel products that (1) contain silicone in the oil phase and (2) does not control the propylene glycol content.

U.S. Pat. No. 5,925,338 issued to Gillette describes a clear gel comprising selected amounts of various types of silicones.

U.S. Pat. No. 6,419,910 assigned to Unilever describes a clear emulsion and gel-type antiperspirant and deodorant composition which comprises a water in oil emulsion which is essentially free of glycols and low and middle chain alcohols. The composition comprises 25-35% of an oil phase comprising at least one non-volatile ester or at least one nonvolatile silicone wherein at least one oil phase soluble ingredients has a refractive index of about 1.40 to about 1.45. The active phase contains a water soluble, non-simple glycol component which raises the refractive index of the aqueous solution.

U.S. Pat. No. 6,410,002 and U.S. patent application Ser. No. 2002/10051138 A1 also assigned to Unilever, describes an essentially glycol free clear emulsion and gel-type antiperspirant and deodorant composition in which the water phase further differentiated by containing at least one polymeric ethylene oxide glycol and is essentially free of glycols and low and middle chain alcohols.

U.S. Pat. No. 6,042,816 describes enhanced efficacy antiperspirant salt compositions containing calcium and an amino acid or a hydroxy acid, methods of making such enhanced efficacy antiperspirant salt compositions, stabilized aqueous solutions of such enhanced efficacy antiperspirant salt compositions, and topical compositions containing such enhanced efficacy antiperspirant salt compositions.

U.S. Pat. No. 6,468,512 assigned to Avon describes a clear antiperspirant/deodorant gel composition. The composition is a water-in-oil emulsion having a viscosity about 7,000 cps to about 25,000 cps and a clarity from about 30 NTU or less. The composition further has an antiperspirant active, water, silicone gelling agent, and one or more silicone oils.

U.S. Pat. No. 6,485,716 assigned to the same owner as the present case describes a clear, elastomer-free, gel composition comprising: (a) 0.1-25 weight % of an antiperspirant active having a low metal to chloride ratio in the range of 0.9-1.3: 1; (b) 9-23.95 weight % of one or more volatile silicones having a flash point of 100 degrees C. or less; (c) 0.05-0.5 weight % of a silicone surfactant having an HLB value less than or equal to 8; (d) 30-70 weight % water; (e) 0-50 weight % selected water soluble organic solvents; and (f) 0-10 weight % of an emollient; wherein the composition is a liquid gel having a viscosity in the range of 5-50,000 centipoise and a ratio of oil phase to water phase in the range of 10:90 to 24:76.

U.S. Pat. No. 6,500,412 assigned to the same owner as this case describes a non-sticky, clear water-in-oil emulsion comprising: (a) 65-90 weight % of an internal phase comprising 5-35 weight % of an antiperspirant salt (anhydrous basis) having a metal:chloride ratio in the range of 0.9-1.4:1; 5-15 weight % of tripropylene glycol; and 35-70 weight % water; and (b) 10-35 weight % of an external phase comprising 1-40 weight % of a volatile silicone which is not an elastomer; 0.1-5 weight % of a silicone copolyol surfactant; and 0-20 weight % of a nonvolatile silicone which is not an elastomer; wherein the composition is free of (1) C1-5 saturated alcohols, (2) added propylene glycol, (3) elastomer gelling agents, (4) soap gelling agents (5) borate gelling agents, and (6) coupling agents, and wherein all amounts are in % by weight based on the total weight of the composition.

While various cosmetic gel compositions, including antiperspirant and deodorant compositions that are clear are known, it is still desired to provide a cosmetic cream composition (e.g., clear antiperspirant and/or deodorant cream composition) which has improved efficacy in comparison to other products, especially other commercially available gel products. It is a further object of the invention to provide products which have (a) reduced whitening, (b) low tack, (c) a quick dry down profile and (d) reduced skin irritation potential relative to commercially available products. This invention has an oil phase which has a relatively low refractive index, thereby reducing the level of water soluble organic or silicone based refractive index matching agents (such as glycols and other monohydric or polyhydric alcohols, ionizable monovalent or divalent inorganic salts, sugars, esters and amino acids) used to match the refractive index of the water (internal) phase to the oil (external) phase to obtain a clear gel.

It is an object of the present invention to provide suspension free creams which impart a superior non-greasy sensory feel when compared to the antiperspirant creams currently marketed.

It is another object of the present invention to provide a cream composition which goes on clear when applied to the skin and which does not clump in the underarm hair.

It is still a further object of the present invention to provide an efficacious suspension free cream composition which has at least equal antiperspirant efficacy to a clear water in oil emulsion gel with improved skin feel aesthetics due among other factors, to its lower level of refractive index matching agents in the active phase such as glycols, and polymeric ethylene oxide glycol adducts.

The cream consists of a mixture of an oil phase and a water phase with a white appearance due to with mismatched refractive indexes. The actual color can be manipulated using a variety of techniques which include but are not limited to, the use of dyes and use of refractive agents, etc. For instance a light blue cream can be made by adding a oil soluble or water soluble blue dye to the white suspension free cream composition wherein the intensity or shade of the color is dependent on the amount of dye added and the turbidity of the cream composition without dye.

### SUMMARY OF THE INVENTION

The invention is a white (defined as not allowing light to pass through the composition and quantified as having turbidity>350 NTU at 21.0 degrees C.), high efficacy, elastomer free, high viscosity (>150,000 centipoise) water-in-oil emulsion which forms a suspension free cream. These creams comprise a glycine containing antiperspirant active with a low metal to chloride ratio in a high water content (>30 weight % of the formula) internal (aqueous) phase, a copolyol, and a fragrance solubilizer in the external phase. The external (oil) phase of the composition contains silicone based emollients, both volatile and nonvolatile with no limitations set on their refractive indices. The internal (water) phase is free of any secondary emulsifiers or surfactants having an HLB value between 9 and 18 inclusive. Monovalent or divalent salts are added in the aqueous phase, particularly in compositions where the antiperspirant level (anhydrous) has been reduced to less than 17% to add stability to the composition. The components that comprise the water phase and the oil phase can be optimized for skin feel and efficacy without the restrictions imposed by requiring a clear system.

While in clear gels the requirements needed for refractive index matching limit the composition ranges and options for different ingredients, in the present invention the white cream allows more flexibility in formulations to provide improved sensory, aesthetics and fragrance hedonics while maintaining efficacy. For example, in a clear composition, an increase in the amount of fragrance has to be followed by an increase in the amount of fragrance solubilizer, e.g. PPG 3, which leads to a two-fold increase in the refractive index of the oil phase. To maintain the overall clarity of the gel, the refractive index of the water phase needs to be readjusted with higher glycol levels. This results in less desirable skin feel such as slow dry-down and tackiness. Such problems are alleviated in the cream composition described in the present invention.

The suspension free cream compositions of this invention can include increased amounts of the cosmetically active ingredient (for example, increased amounts of antiperspirant active ingredient), and yet can be effective with low levels of active as well. These suspension free cosmetic creams are quick drying due to the combination of low levels of nonvolatile materials in the oil phase and low levels (less than 3%) of glycols.

### DETAILED DESCRIPTION OF THE INVENTION

The invention is a white, elastomer-free, suspension free, emulsion having an external oil phase and internal water phase wherein:

(I) the oil phase comprises:

(a) 7.0-28.4 weight % (more particularly 9-20 weight %) of one or more cyclomethicones having a flash point of 100 degrees C. or less;

(b) 0.6-2.0 weight % on an active basis (particularly 0.6-1.0% ) of a silicone surfactant having an HLB value (hydrophilic lipophilic balance)≦8);

(c) 1-5 weight % (particularly 1-2.5 weight %) of a silicone based emollient having a refractive index in the range of 1.4000 to 1.4800;

(d) 0-3.0 weight % of an non-siliconized organic fragrance solubilizer (particularly 0.5-2%) consisting of silicone compatible straight or branched hydrocarbons with a molecular weight less than 1000, alkyl substituted phenyl esters with an alkyl carbon chain length between C-1 to C-20, and ethoxylated and or propoxylated ethers with a carbon chain length from C-1 to C-25 and ethoxylation and or propoxylation from 1-10 (for example, a member of the group consisting of hydrogenated polyisobutene (Polyiso 250), C12-15 alkyl benzoate (FINSOLV TN), and PPG-3 myristyl ether (particularly hydrogenated polyisobutene (Polyiso 250), C12-15 alkyl benzoate (FINSOLV TN), and PPG-3 myristyl ether, and, most particularly, PPG-3 myristyl ether) which can help to solubilize the fragrance oils in the otherwise all silicone based oil phase and which does not negatively affect the skin feel and dry down characteristics of the composition. (Note that aesthetic skin feel attributes such as wetness, stickiness, and residue have been evaluated by a 10 member trained panel both on the forearm and underarm by rating some of the antiperspirants described in this invention along with a commercial gel. Significant differences in performance between some of the formulas described herein and a commercial gel were noted at the 95% confidence level.);

(e) 0-5 weight % fragrance or odor masking component; and

(II) the aqueous phase comprises:

(a) 0.1-30 weight % on an anhydrous basis (particularly 17-30 weight % and, more particularly, 15-20 weight %) of a glycine containing antiperspirant active salt comprising either aluminum or aluminum and zirconium metals such that (i) if aluminum and zirconium salt is used then the metal/Cl ratio of the salt should be low, such as 0.9-1.5:1 (and preferably of 0.9-1.05:1); the glycine/Zr ratio should be >1.0. (ii) if aluminum salt is used then the aluminum to chloride molar ratio should be in the range of 0.5-2.5:1; the glycine/Al molar ratio should be in the range of 0.05-0.26:1 (preferably in the range of 0.05-0.16:1); wherein the glycine containing antiperspirant active salt has a pH in the range of 2-4 (when measured in water at a concentration of 15%), is free of any other halide scavenging material.

(b) 30-70 weight % water (particularly 45-65% and, more particularly, 50-60%);

(c) 0.2-4 weight % of a monovalent or divalent ionizable, water-soluble inorganic or organic salts to help increase the refractive index of the active phase and optimize the glycol level. These salts are of the form MₐX_{b} where a=1, or 2 and b=1 or 2; M is a member selected from the group consisting of Na⁺¹, Li⁺¹, K⁺¹, Mg⁺², Ca⁺², Sr⁺² and Zn⁺² and X is a member selected from the group consisting of chloride, bromide, iodide, citrate, gluconate, lactate, glycinate, glutamate, ascorbate, aspartate, nitrate, phosphate, hydrogenphosphate, dihydrogenphosphate, formate, maloneate, maleate, succinate, carbonate, bicarbonate, sulfate, hydrogensulfate. Salts of particular utility are NaCl and ZnCl₂;

(d) 0-3.0 weight % of a water soluble glycol system selected from the group consisting of propylene glycol ethylene glycol; diethylene glycol; triethylene glycol; tetraethylene glycol; propylene glycol; dipropylene glycol; tripropylene glycol; 1,3 propanediol; 2-methyl propanediol; methyl propanediol; 1,6-hexanediol; 1,3 butanediol; 1,4 butanediol; PEG-4 through PEG-600; PPG-9 through PPG-34; neopentyl glycol; trimethylpropanediol; 2,2 dimethyl-1,3propandiol; 2,2,4,4-tetramethyl-1,3-cyclobutane-diol; and mixtures thereof (More particular examples of the glycol component include one or more members of the group consisting of propylene glycol, dipropylene glycol, 2-methyl-1,3 propanediol, methyl propylene glycol, low molecular weight (less than 600) polyethylene glycol and mixtures of any of the foregoing.);

(e) 0-5% of a water soluble carbon based emollient other than glycols which includes but is not limited to glycerin;

(f) 0-10% trimethylglycine (“Betaine,”) wherein the Betaine:aluminum molar ratio is in the range of 0.05-0.50:1.0 and, if the antiperspirant active contains zirconium, the Betaine/zirconium molar ratio is in the range of 0.2-3.0:1; and

(g) 0-10 weight % (particularly 0-4%) of an alcohol having 2-4 carbons (for example, ethanol);

wherein (i) all amounts are based on the percent weight of the entire composition, (ii) the composition is a water in oil emulsion having a viscosity greater than 150,000 centipoise (for example, in the range of 150,000-600,000 centipoise, more specifically, 200,000-350,000 centipoise), and (iii) the composition has a ratio of oil phase to water phase in the range of 10:90 to 30:70.

According to a first aspect of the present invention, various of the foregoing objects are achieved through a high viscosity emulsion having (1) an aqueous phase containing water (or water and a water soluble organic solvent as defined above); the antiperspirant active containing aluminum and zirconium metals having a M:Cl ratio (0.9 to 1.5:1; and at least one monovalent or divalent salt, a glycol containing two hydroxy groups or polymeric glycol in which the total level of glycol species does not exceed 3 weight percent; and (2) an oil phase containing a volatile organic or silicone material, and the composition further including (3) a suitable silicone based surfactant such as an alkoxylated, alkyl substituted siloxane surface active agent in an amount of 0.6-0.9 weight % (on an actives basis) suitable to form a high viscosity (>150,000 cps) cream as described above; (4) at least 1% of a silicone emollient; and (5) a suitable fragrance solubilizer such as myristyl ether which helps to solubilize the fragrance oils in the predominantly silicone based oil phase.

The refractive index of the active phase is mismatched to the oil phase (comprised of fragrance, surfactants, fragrance solubilizers and silicones) by at least 0.0050 by minimizing the level of glycols and salts, whereby the criteria used to determine the level of active phase ingredients is based on skin aesthetics and efficacy not on the ability to obtain a clear product. By eliminating the clarity criterion (matching the refractive index of the oil and water phases to within 0.0040 units, preferably within 0.020 units), an aesthetically more pleasing cream can be obtained while maintaining efficacy.

Refractive index measurements are made at a temperature of about 20-25 degrees C using a Bausch and Lomb Abbe 3L Refractometer. Turbidity measurements as described herein are made with an Orbeco-Hellige #965 Direct-Reading Turbidimeter.

One embodiment of the invention is comprised of an oil phase composition such that the addition of the fragrance component (if fragrance is added to the composition) provides a refractive index in the range from about 1.4015 to about 1.4150; especially from about 1.4025 to about 1.4090.

One of the benefits of adding a non-siliconized organic fragrance solubilizer is to improve the solubility of the fragrance within the silicone (primarily cyclomethicones) based oil phase of the emulsion. The limited solubility of some fragrances in the cyclomethicone and linear polydialkylorganosiloxanes (if present) is easily determined by the turbidity of the aforementioned silicone(s) when approximately 10-30 weight % of fragrance is added to the silicone. To those skilled in the art, it is known that the addition of fragrance solubilizer such as PPG-3 myristyl ether (the amount depending on the fragrance oil) is paramount to ensure clarity within a clear gel. In the present invention these restrictions are eliminated and therefore the fragrance solubilizer is optional (0-3%).

The high viscosity suspension free cream compositions of the present invention include an antiperspirant active agent in an amount sufficient to have a deodorizing effect and/or in an amount sufficient to reduce the flow of perspiration when the composition is applied to a human. For the antiperspirant active used in the internal (also called “active”) phase various antiperspirant active materials that can be utilized according to the present invention provided that they are soluble at a suitable concentration in the active phase.

Antiperspirant actives can be incorporated into compositions according to the present invention in amounts, such as in the range of 7-25% (on an anhydrous solids basis), preferably 7-20%, by weight, of the total weight of the composition. Mixtures of actives can also be used. The amount used will depend on the formulation of the composition. At amounts at the higher end of the range (especially in a range of 9-20% or 9-25%, a good antiperspirant effect can be expected. As noted above, the active is preferably included in the compositions of the invention by premixing the active with water and possibly small amount of propylene glycol.

Antiperspirant actives can also be incorporated into compositions according to the present invention in amounts in the range of 0.1-25% of the final composition, but the amount used will depend on the formulation of the composition. For example, at amounts in the lower end of the broader range (for example, 0.1-9% on an actives basis), a deodorant effect may be observed. At lower levels the antiperspirant active material will not substantially reduce the flow of perspiration, but will reduce malodor, for example, by acting as an antimicrobial material. At amounts of 9-25% (on an actives basis) such as 15-25%, by weight, of the total weight of the composition, an antiperspirant effect may be observed.

The antiperspirant active materials disclosed therein, including the acidic antiperspirant materials, can be incorporated in the compositions of the present invention if they are soluble in the active phase. Suitable materials include (but are not limited to) aluminum chlorides (various types including, for example, anhydrous form, hydrated form, etc.), zirconyl hydroxychlorides, zirconyl oxychlorides, basic aluminum chlorides, basic aluminum chlorides combined with zirconyl oxychlorides and hydroxychlorides, and organic complexes of each of basic aluminum chlorides with or without zirconyl oxychlorides and hydroxychlorides and mixtures of any of the foregoing. These include, by way of example (and not of a limiting nature), aluminum chlorohydrate, aluminum chloride, aluminum sesquichlorohydrate, aluminum chlorohydrol-propylene glycol complex, zirconyl hydroxychloride, aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly), aluminum dichlorohydrate, aluminum chlorohydrex PG, aluminum chlorohydrex PEG, aluminum dichlorohydrex PG, aluminum dichlorohydrex PEG, aluminum zirconium trichlorohydrex gly propylene glycol complex, aluminum zirconium trichlorohydrex gly dipropylene glycol complex, aluminum zirconium tetrachlorohydrex gly propylene glycol complex, aluminum zirconium tetrachlorohydrex gly dipropylene glycol complex, and mixtures of any of the foregoing. The aluminum-containing materials can be commonly referred to as antiperspirant active aluminum salts. Generally, the foregoing metal antiperspirant active materials are antiperspirant active metal salts. In the embodiments which are antiperspirant compositions according to the present invention, such compositions need not include aluminum-containing metal salts, and can include other antiperspirant active materials, including other antiperspirant active metal salts. Generally, Category I active antiperspirant ingredients listed in the Food and Drug Administration's Monograph on antiperspirant drugs for over-the-counter human use can be used. In addition, any new drug, not listed in the Monograph, such as tin or titanium salts used alone or in combination with aluminum compounds (for example, aluminum-stannous chlorohydrates), aluminum nitratohydrate and its combination with zirconyl hydroxychlorides and nitrates, can be incorporated as an antiperspirant active ingredient in antiperspirant compositions according to the present invention. Preferred antiperspirant actives that can be incorporated in the compositions of the present invention include the enhanced efficacy aluminum salts and the enhanced efficacy aluminum/zirconium salt-glycine materials, having enhanced efficacy due to improved molecular distribution, known in the art and discussed, for example, in PCT No. WO92/19221, the contents of which are incorporated by reference in their entirety herein. Particular actives include Westchlor A2Z 4105 aluminum zirconium tetrachlorohydrex gly propylene glycol complex, (from Westwood Chemical Corporation, Middletown, N.Y.); Westchlor ZR 35B aluminum zirconium tetrachlorohydrex gly, and Rezal 36 GP and AZP 902 aluminum zirconium tetrachlorohydrex gly both from Reheis, Berkeley Heights, N.J. as well as Rezal AZZ 908 from Reheis. In general, the metal:chloride mole ratio is in the range of 2.1-0.9:1 for such salts.

Particularly preferred are antiperspirant actives having the specific low metal to chloride ratio specified above and those described in U.S. Pat. No. 6,375,937 and a patent application assigned to the same owners as this case (Attorney Docket number IR 6997, U.S. Ser. No. 10/314,712 filed Dec. 9, 2002).

In one particular type of salt of interest, an aluminum zirconium tetrasalt with glycine is used wherein aluminum zirconium tetrachlorohydrex glycine salt having a metal to chloride ratio in the range of 0.9 to 1.5:1 and a glycine:Zr mole ratio>1.0.

One embodiment of this invention uses an antiperspirant salt with a metal:chloride ratio of 0.9 to 1.2:1 (especially in the range of 0.9 to 1.1:1 and, more particularly in the range of 0.9 to 1.0:1); and a glycine:zirconium mole ratio greater than 1.2:1. This type of salt may be made in a variety of ways as described in U.S. Pat. No. 6,375,937 as referenced above.

Another particular type of salt of interest is an aluminum chloride salt buffered by glycine, wherein the salt has a metal to chloride ratio in the range of 0.9 to 1.2:1 (especially in the range of 0.9 to 1.1:1 and, more particularly in the range of 0.9 to 1.0:1). Also of interest are salts which include Betaine, additional glycine, or another amino acid such as alanine to further increase the refractive index of the glycine-containing active complex.

Examples of salts include those made as follows:

Method A: An aluminum chlorohydrate (ACH) solution of ACH salt in water of suitable concentration is mixed with an aqueous solution of zirconyl chloride (ZrOCl₂) of suitable concentration and powdered glycine. The mixture is stirred at room temperature to obtain the salt.

Method B: A suitable commercially available aluminum zirconium tetrachlorohydrex glycine salt is obtained and mixed with a sufficient amount of an aqueous aluminum chloride (AlCl₃) solution and powdered glycine. The mixture is stirred at room temperature to obtain the salt. When Method B is used, a suitable salt to use as a starting material includes various types of tetra salts such as aluminum zirconium tetrachlorohydrex gly, aluminum zirconium tetrachlorohydrex gly propylene glycol complex, aluminum zirconium tetrachlorohydrex gly dipropylene glycol complex, and mixtures of any of the foregoing. These salts may be referred to hereinafter as experimental salts or carry an “exp” suffix in their designation. It is preferred that the experimental salt be used in the form of a 28-50% water solution when added to form the compositions of the invention.

Suitable silicone surfactants include silicone polyglucosides (for example, octyl dimethicone ethoxy glucoside) and silicone copolyols having an HLB value (hydrophilic lipophilic balance)≦8. The HLB value may be measured in a variety of ways such as described in conventional references or found listed in tables of data recording such values. It is intended that any type of HLB measurement technique may be used.

A silicone copolyol (especially dimethicone copolyol) may be used in an amount of 0.6-0.9 weight % (actives basis), particularly 0.6-1.0.

In general, silicone copolyols useful in the present invention include copolyols of the following Formulae I and II. Formula I materials may be represented by:

(R¹⁰)₃—SiO—[(R¹¹)₂—SiO]ₓ—[Si(R¹²)(R^{b}—O—(C₂H₄O)ₚ—(C₃H₆O)ₛ—R^{c})O]_{y}—Si—(R¹³)₃ Formula I

wherein each of R¹⁰, R¹¹, R¹² and R¹³ may be the same or different and each is selected from the group consisting of C1-C6 alkyl; R^{b} is the radical —CₘH₂ₘ—; R^{c} is a terminating radical which can be hydrogen, an alkyl group of one to six carbon atoms, an acyl group such as an ester with a terminating alkyl group of 1-4 carbons, or an aryl group such as phenyl; m has a value of two to eight; p and s have values such that the oxyalkylene segment —(C₂H₄O)ₚ—(C₃H₆O)ₛ— has a molecular weight in the range of 200 to 5,000; the segment preferably having fifty to one hundred mole percent of oxyethylene units —(C₂H₄O)ₚ— and one to fifty mole percent of oxypropylene units —(C₃H₆O)ₛ—; x has a value of 8 to 400; and y has a value of 2 to 40. Preferably each of R¹⁰, R¹¹, R¹² and R¹³ is a methyl group; R^{c} is H; m is preferably three or four whereby the group R^{b} is most preferably the radical —(CH₂)₃—; and the values of p and s are such as to provide a molecular weight of the oxyalkylene segment —(C₂H₄O)ₚ—(C₃H₆O)ₛ— of between about 1,000 to 3,000. Most preferably p and s should each have a value of about 18 to 28.

A second siloxane polyether (copolyol) has the Formula II:

(R¹⁰)₃—SiO—[(R¹¹)₂—SiO]ₓ—[Si(R¹²)(R^{b}—O—(C₂H₄O)ₚ—R^{c})O]_{y}—Si—(R¹³)₃ Formula II

wherein p has a value of 6 to 16; x has a value of 6 to 100; and y has a value of 1 to 20 and the other moieties have the same definition as defined in Formula I.

It should be understood that in both Formulas I and II shown above, that the siloxane-oxyalkylene copolymers of the present invention may, in alternate embodiments, take the form of endblocked polyethers in which the linking group R^{b}, the oxyalkylene segments, and the terminating radical R^{c} occupy positions bonded to the ends of the siloxane chain, rather than being bonded to a silicon atom in the siloxane chain. Thus, one or more of the R¹⁰, R¹¹, R¹² and R¹³ substituents which are attached to the two terminal silicon atoms at the end of the siloxane chain can be substituted with the segment —R^{b}—O—(C₂H₄O)ₚ—(C₃H₆O)ₛ—R^{c} or with the segment —R^{b}—O—(C₂H₄O)ₚ—R^{c}. In some instances, it may be desirable to provide the segment —R^{b}O—(C₂H₄O)ₚ—(C₃H₆O)ₛ—R^{c} or the segment —R^{b}—O—(C₂H₄O)ₚ—R^{c} at locations which are in the siloxane chain as well as at locations at one or both of the siloxane chain ends.

Particular examples of suitable dimethicone copolyols are available either commercially or experimentally from a variety of suppliers including Dow Corning Corporation, Midland, Mich.; General Electric Company, Waterford, N.Y.; Witco Corp., Greenwich, Conn.; and Goldschmidt Chemical Corporation, Hopewell, Va. Examples of specific products include DOW CORNING® 5225C from Dow Corning which is a 10% dimethicone copolyol in cyclomethicone; DOW CORNING® 2-5185C which is a 45-49% dimethicone copolyol in cyclomethicone; SILWET L-7622 from Witco; ABIL EM97 from Goldschmidt which is a 85% dimethicone copolyol in D5 cyclomethicone; and various dimethicone copolyols available either commercially or in the literature.

It should also be noted that various concentrations of the dimethicone copolyols in cyclomethicone can be used. While a concentration of 10% in cyclomethicone is frequently seen commercially, other concentrations can be made by stripping off the cyclomethicone or adding additional cyclomethicone. The higher concentration materials such as DOW CORNING® 2-5185 material is of particular interest.

In one particular embodiment 3-9 weight % (particularly 5-8%) of a 10% silicone copolyol such as dimethicone copolyol in cyclomethicone mixture may be used, wherein the amount of mixture added is selected so that the level of silicone copolyol in the cosmetic composition is in the range of 0.6-0.9% (particularly 0.6-0.8%) (for example, 6.5% of a 10% dimethicone copolyol in cyclomethicone mixture).

The cyclomethicones used in this invention are one or more members selected from the group consisting of cyclic polydimethylsiloxanes such as those represented by Formula III:

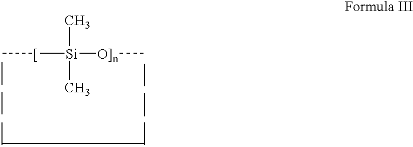

where n is an integer with a value of 4-6, particularly 5-6. These include a tetramer (D4), a pentamer (D5), and a hexamer (D6), and mixtures of any two or three of the forgoing. For example, DC-245 fluid and DC-345 from Dow Corning Corporation (Midland, Mich.) are types of cyclomethicones which can be used. It is to be noted that for purposes of the present invention cyclomethicones are not considered as silicone emollients.

Emollients are a known class of materials in this art, imparting a soothing effect to the skin. Emollient selection is limited to silicone based emollients and is required at a level of at least 1 weight percent for the purpose of the present invention. Emollients are ingredients which help to maintain the soft, smooth and pliable appearance of the skin. Emollients are also known to reduce whitening on the skin and/or improve aesthetics.

Particular examples of suitable emollients include members of the group consisting of linear silicones (both volatile and non-volatile) such as linear dimethicones, particularly dimethicones having a viscosity in the range of 0.5-1000 centistokes, indicated in Formula IV.

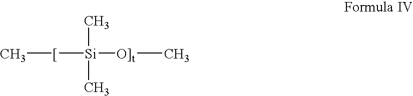

Further examples include Dow Corning® DC 200, dimethiconols (such as but not limited to Dow Corning® DC1501 ), Dow Corning 2501 cosmetic wax (dimethicone copolyol) dimethiconol behenate, C₃₀₋₄₅ alkyl methicone, stearoxytrimethylsilane, and stearyl dimethicone) as well as silanol DC 9023 also from Dow Corning). Another example of a suitable silicone emollient is phenyl trimethicone (DC 556 also from Dow Corning).

The oil phase according to the present invention is, desirably, a silicone oil phase, so as to provide a water-in-silicone oil emulsion. The total of oil phase and siloxane surface-active agent preferably makes up from about 10% to about 24% by weight, of the total weight of the composition. This surface-active agent is an emulsifier which, when properly mixed with the aqueous phase components, and oil phase components, yields a water-in-oil emulsion. The oil phase is desirably a blend of liquids, but does not contain any significant amount of non-volatiles (that is, less than 5.0 weight % of any material having a flash point greater than 100 degrees C.).

The oil phase can include, illustratively, a volatile silicone fluid such as one or more of D4, D5 and D6 cyclomethicones, as well as phenyl dimethicone. Where the composition includes the volatile silicone, it is preferred that such volatile silicone be a polydimethylcyclosiloxane, present in an amount up to about 28.4% by weight, of the total weight of the composition, preferably from about 7% to about 20% by weight, of the total weight of the composition. Preferred polydimethylcyclosiloxanes are those named cyclomethicones, exemplified by the formula ((CH₃)₂ SiO)ₓ where x is a number from about 4 to about 6. Preferred cyclosiloxanes are octamethylcyclotetrasiloxane (x=4), decamethylcyclopentasiloxane (x=5) and blends of tetramer and pentamer cyclomethicones. Commercial cyclosiloxanes which can be utilized as part of the composition of the present invention include, illustratively, Dow Corning 244 fluid, Dow Corning 245 fluid, Dow Corning 344 fluid and Dow Corning 345 fluid (from Dow Corning Corp.).

Preferably the oil phase is a mixture of volatile silicone fluids such as one or more of D4, D5 and D6 cyclomethicones, especially D5 and D6 cyclomethicones.

A particular example of an alkoxylated, alkyl substituted siloxane surface active agent is preferably, but not limited to, a dimethicone copolyol. An illustrative alkoxylated silicone-containing surfactant utilizable according to the present invention is cetyl dimethicone copolyol, referred to in U.S. Pat. No. 5,162,378 to Guthauser. Illustratively, the alkoxylated, alkyl substituted siloxane surface active agent is included in the composition in an amount of 6.0% to 9.0% by weight, of the total weight of the composition. Another example of a suitable surfactant is octyl dimethicone ethoxy glucoside (from Wacker-Belsil, Adrian, Mich.).

A specific cyclomethicone-dimethicone copolyol fluid which can be utilized to provide the alkoxylated silicone-containing surface-active agent is a mixture of cyclomethicone and dimethicone copolyol designated as DC 5225C from Dow Corning Corporation. This is a polyether substituted silicone of cyclomethicone and dimethicone copolyol (refractive index (RI)=1.3994) at about 20-25 degrees C. This DC 5225C, which is an emulsifying agent, is useful for preparing stable water-in-oil emulsions where a silicone makes up a large portion of the oil phase, and is a dispersion of a silicone surfactant (dimethicone copolyol) (10% by wt.) in cyclomethicone (Dow Corning 245) (90% by weight).

The mixture of cyclomethicone and dimethicone copolyol fluid is present in the composition, illustratively, in an amount of from about 7.0% to about 28.4% by weight, of the total weight of the composition.

According to another aspect of the present invention, the aqueous phase of the cosmetic cream composition further includes a glycol system in low amount (0-3% preferably 0.3-1.5%) comprising a glycol or polyglycol or combination thereof for optimizing the cosmetic properties, including a reduction of tack and a decrease in the whitening and in the residue after application of the composition. Moreover, compositions incorporating polypropylene glycol, particularly, tripropylene glycol, have improved mildness (that is, reduced skin irritation potential) relative to commercially available products. The glycol or polyglycol is selected from the group consisting of ethylene glycol, propylene glycol, 1,2 propanediol, 2-methyl propanediol, diethylene glycol, triethylene glycol, tetraethylene glycol, dipropylene glycol, tripropylene glycol, methyl propanediol, 1,6-hexanediol, 1,3 butanediol, 1,4 butanediol, PEG-4 through PEG-600, PPG-9 through PPG-34, neopentyl glycol, trimethylpropanediol, 2,2 dimethyl-1,3 propanediol, 2,2,4,4-tetramethyl-1,3-cyclobutane-diol and mixtures thereof. More particular examples of the glycols which can be used with the propylene glycol are one or more members of the group consisting of dipropylene glycol, 2-methyl-1,3 propanediol, methyl propylene glycol, low molecular weight (less than 600) polyethylene glycol and mixtures of any of the foregoing.

The cream compositions of the invention further include at least one ionizable inorganic salt which helps to stabilize the cream particularly when low levels of antiperspirant salt are used. These ionizable salts are of the form MₐX_{b} where a=1, or 2 and b=1 or 2; M is a member selected from the group consisting of Na⁺¹, Li⁺¹, K⁺¹, Mg⁺², Ca⁺², Sr⁺² and Zn⁺² and X is a member selected from the group consisting of chloride, bromide, iodide, citrate, gluconate, lactate, glycinate, glutamate, ascorbate, aspartate, nitrate, phosphate, hydrogenphosphate, dihydrogenphosphate, formate, maloneate, maleate, succinate, carbonate, bicarbonate, sulfate, hydrogensulfate. Salts of particular utility are NaCl and ZnCl₂. As will be appreciated by those skilled in the art, while it may be possible under certain circumstances to add a salt directly to a portion of the mixture during manufacturing, it is preferred to add the salt as a mixture or solution of the salt in a carrier or solvent, particularly water. Of course various concentrations of the salt can be made such as in the range of 1-40%.

The present invention also includes methods of forming high viscosity cosmetic cream compositions described herein. In such methods an aqueous phase comprising water and the antiperspirant active is formed separately from the oil phase containing an alkoxylated, alkyl substituted siloxane surface active agent, an organic fragrance solubilizer and cyclomethicone and optional fragrance. The two phases are then combined and homogenized to achieve the desired viscosity.

For one embodiment the active phase is a water phase containing 15-30 weight % (anhydrous) of the low metal:chloride glycine-containing antiperspirant active and 0.5-3 weight % of the water-soluble glycol system and the monovalent or divalent ionizable water-soluble salt. Illustratively, free water is included in the composition in the range of 30-70 weight % based on the total weight of the composition. The water phase can optionally include, for example, (along with the antiperspirant active, water-soluble glycol system and ionizable divalent or monovalent salt) 0-10 weight % of a water-soluble carbon-based, emollient and/or Betaine.(as defined above, trimethylglycine).

Preparation of the suspension free cream composition is greatly simplified since refractive index matching is not needed. In fact, active and oil phases which are formulated to give clear products frequently reflect a compromise in aesthetics. Not only do the compositions of this invention avoid this problem, they may be formulated to give further whiteness for a more pleasing visual effect. This may be done by minimally readjusting the ingredients in the oil and/or water phases to achieve a greater refractive index mismatch.

Elimination of the clarity criterion also allows for greater formula flexibility with respect to fragrancing. Fragrances typically have a high refractive index (usually greater than 1.43) and are best kept within the oil phase so as to avoid any possible interaction and potential degradation with the acidic antiperspirant active. Increasing the level of fragrance results in an increase in the refractive index of the oil phase. With a clear gel this would require adding additional refractive index matching agents within the active phase thereby potentially negatively impacting the skin feel. For the suspension free cream compositions of the present invention, the fragrance level can be dialed up or down without having to alter the active phase composition.

The suspension free water-in-oil cream is prepared by adding the aqueous phase to the oil phase (for example, the aqueous phase is slowly added to the oil phase with turbulent agitation), and then additional additives, or other active ingredients, are added with mixing. Alternatively, the fragrance can be added after the active phase is added to the oil phase. The resulting cream emulsion is then passed through, for example, a colloid mill or other high shear emulsifier so as to provide a viscous product which can be transferred to a suitable applicator or container for use by the consumer.

These compositions of the present invention may be prepared by a batch process, or a continuous or semi-continuous process, and the processes yield compositions which are stable, highly efficacious and possess excellent aesthetic qualities.

The compositions according to the present invention are used as conventional cosmetic cream compositions. For example, where the composition according to the present invention is cream composition having a viscosity greater than 150,000 cps, packaged in a dispensing container having a top surface with slots or pores, the suspension free cream is extruded from the dispensing container through the slots or pores and applied to the skin (for example, in axillary regions of the human body) by rubbing the soft cream material extruded through the top surface of the container on the skin in the axillary region.

As a further aspect of the present invention, the dispensing container can be clear and can be tinted so as to for example, fit to the fragrance hedonics. The composition has reduced tack, quick dry down, a cool sensation, and a silky feel and imparts much less or no white residue on dry down compared to commercially available products. Moreover, compositions of the present invention incorporating a polypropylene glycol component have improved mildness (have reduced skin irritation potential) as compared to commercially available products, and have improved cosmetic properties (including reduced tackiness) and reduced white residue upon application. The cream emulsions according to the present invention are stable, cosmetically elegant, and are capable of being delivered from a suitable applicator package.

Throughout the present specification, the antiperspirant active materials, when utilized in an antiperspirant effective amount in the composition, act to reduce body malodor by reducing production of perspiration; however, these antiperspirant active materials can also have a deodorant function, e.g., as an antimicrobial agent. The deodorant active materials do not substantially reduce the production of perspiration, but reduce malodor in other ways, e.g., as fragrances masking the malodor or reducing the malodor intensity, as odor absorbents, as antimicrobial agents, as agents chemically reacted with malodorous materials, etc.

The amount of active component that can be used will vary with the particular active ingredient incorporate. The product comprises antiperspirant active materials in amounts sufficient to combat body malodor either as a deodorant or as an antiperspirant when applied to the axillary regions of the body. As a general rule, an antiperspirant product should contain an active antiperspirant material in an amount anywhere from for example, about 7% to about 25% by weight, of the total weight of the composition. The active antiperspirant material utilized in the compositions of the present invention can be pre-dissolved in water or in another solvent (for example, in propylene glycol), and may be buffered or unbuffered. Preferably, the antiperspirant materials are present in solution in a solvent.

Where a deodorant active material is utilized other than lower amounts of an antiperspirant active (which can be used in amounts in the range of 0.1-7.0 weight %), any deodorant active material, which can be dissolved in the oil phase, can be utilized in an amount sufficient to have a deodorant effect. Illustratively, the deodorant active material can be 2,4,4′-trichloro-2′-hydroxy diphenyl ether (triclosan), and/or benzethonium chloride and/or octoxyglycerin (Sensiva® SC 50). Where the deodorant ingredient is used in place of the antiperspirant active ingredient, a deodorant cream composition (rather than an antiperspirant gel composition) would be provided.

Throughout the present specification, where compositions are described as including or comprising specific components or materials, it is contemplated by the inventors that the compositions of the present invention also consist essentially of, or consist of, the recited components or materials. Accordingly, throughout the present disclosure any described composition of the present invention can consist essentially of, or consist of, the recited components or materials.

One particular embodiment is a suspension-free white cream comprising:

(a) 17-23 weight % of an oil phase comprising:

(i) 9-20 weight % of the cyclomethicones;

(ii) 0.6-1.0 weight % on an active basis of the silicone surfactant;

(iii) 1-2.5 weight % of the fragrance solubilizer; and

(iv) 1-2.5 weight % of a silicone emollient; and

(b) 77-83 weight % of an aqueous phase comprising:

(i) 0.1-30 weight % on an anhydrous basis of the antiperspirant active salt;

(ii) 1-3% weight % of the water soluble glycol system selected from the group consisting of propylene glycol, dipropylene glycol, tripropylene glycol, 2-methyl 1,3 propanediol, 1,3 propanediol, methyl propylene glycol, low molecular weight polyethylene glycol;

(iii) 1-4 weight % ethanol or propanol;

(iv) 0.8-3 weight % of the monovalent or divalent ionizable, water soluble inorganic or organic salt;

wherein the amounts are based on the weight of the entire composition.

Various materials incorporated in the water-based phase and in the oil-based phase, and their refractive indices (as measured using the Bausch and Lomb Abbe 3L Refractometer) are set forth in the following particular formulations:

6.0-9.0 weight % dimethicone copolyol/cyclomethicone (10%) (for example, Dow Corning 5225C);

7-28.4 weight % preferably 8-15 weight percent cyclomethicone (D4, D5, D6 or mixtures thereof);

0.0-3.0 weight % PPG-3 myristyl ether;

17-25 weight % antiperspirant active (for example, Al-Zr tetrachlorohydrex gly (such as Z-522, 27.5% from Summit Research Labs, Huguenot, N.Y.) and aluminum dichlorohydrate (such as Westchlor 100, 36.1% to which glycine has been added so that the molar ratio of gly/Al is in the range of 0.05-0.26:1) such as, for example, described in U.S. Pat. No. 6,375,937 and patent application assigned to the same owners as this case (Attorney Docket number IR 6997, U.S. Ser. No. 10/314,712 filed Dec. 9, 2002);

30-70 weight % water;

0.2 to 4.0 weight % of an ionizable salt or combinations of ionizable salts of the form MₐX_{b} where a=1 or 2; b=1 or 2; M is a member selected form the group consisting of Na⁺¹, Li⁺¹, K⁺¹, Mg⁺², Sr⁺² and Zn⁺², Ca⁺² and X is a member selected from the group consisting of chloride, bromide, iodide, citrate, gluconate, lactate, glycinate, glutamate, ascorbate, aspartate, nitrate, phosphate, hydrogenphosphate, dihydrogenphosphate, formate, malonate, maleate, succinate, carbonate, bicarbonate, sulfate and hydrogensulfate (preferred salts are ZnCl₂ and NaCl or combinations thereof);

0-3 weight % of the glycol system as described above in the definition of the invention;

1-5 weight % of a silicone emollients such as dimethiconol, dimethicones, silanol and phenyltrimethicone and combinations thereof in which level of volatile linear silicone components is less than 1.5%;

0-1.0 weight % sage oil (any type such as Clary or Dalmation);

0-5 weight % fragrance or odor masking component;

0-10% of a water soluble non glycol organic solvent selected from the group consisting of 2-4 carbon chain alcohols (for example ethanol),

0-10 weight % of a water-soluble carbon based high refractive index agent such as additional glycine which is not part of the original salt, trimethylglycine, alanine, glycerin, Diglycereth-7, Triglycereth-7 citrate, glycereth-7 glycolate, glycereth-5 lactate, lauramidopropyl glycerin, glycereth-5 lactate, Glycereth-7 glycolate, and glycereth-20 benzoate;

wherein the cream composition has a phase ratio in the range of 10:90-25:75 of oil to water and a viscosity is in the range of 150,000-600-000 centipoise.

### EXAMPLES

The following Examples are offered as illustrative of the invention and are not to be construed as limitations thereon. In the Examples and elsewhere in the description of the invention, chemical symbols and terminology have their usual and customary meanings. In the Examples as elsewhere in this application values for n, m, etc. in formulas, molecular weights and degree of ethoxylation or propoxylation are averages. Temperatures are in degrees C unless otherwise indicated. The amounts of the components are in weight percents based on the standard described; if no other standard is described then the total weight of the composition is to be inferred. Various names of chemical components include those listed in the CTFA International Cosmetic Ingredient Dictionary (Cosmetics, Toiletry and Fragrance Association, Inc., 7^{th} ed. 1997). Refractive Indices (“RI”) are determined at a temperature in the range of 20-25 degrees C.

### Examples 1-10

For Examples shown in Tables A and B the following procedure may be used with the types and amounts of ingredients. The sample sizes are about 500 grams. In the examples where the antiperspirant active is provided as a powder, a pre-mix of active in water is obtained by dissolution in the specified amount of water of the active phase. Silicone copolyol, cyclomethicone and fragrance are weighed and combined in a beaker. The mixture is stirred at 400-600 rpm using a Lightnin Mixer Model L1003. After the mixture becomes visually homogeneous, the active phase containing the antiperspirant active and ionizable salts in water and the rest of the ingredients (propylene glycol and MP diol) are added to the oil phase while mixing. The entire mixture is mixed for 15 minutes. The mixture is then homogenized for 2-4 minutes at a reading of 50-70 on Powerstat Variable Transformer (Superior Electric Co., Bristol, Conn.) using a homogenizer from Greerco Corp. (Hudson, N.H.).

TABLE A

| | EX 1 | EX 2 | EX 3 | EX 4 | EX 5 | EX 6 | EX 7 | EX 8 |
|---|---|---|---|---|---|---|---|---|
| Cyclo- | 9.2 | 7.2 | 9.2 | 11.0 | 9.0 | 9.7 | 9.5 | 10.90 |
| methicone | | | | | | | | |
| Dimethicone | 8.0 | 8.0 | 8.0 | 6.0 | 6.0 | 6.50 | 6.5 | 6.5 |
| copolyol (DC | | | | | | | | |
| 5225C, 10%) | | | | | | | | |
| Dimethiconol | 0.0 | 0.0 | 0.0 | 1.0 | 1.0 | 1.0 | 0.0 | 0.0 |
| DC 1501 | | | | | | | | |
| Dimethicone | 0.0 | 2.0 | 0.0 | 0.0 | 0.0 | 0.0 | 0.0 | 0.0 |
| PPG-3 myristyl | 2.0 | 2.0 | 2.0 | 2.0 | 2.0 | 1.8 | 2.0 | 1.8 |
| ether | | | | | | | | |
| Fragrance | 0.8 | 0.8 | 0.8 | 1.0 | 1.0 | 1.0 | 1.0 | 0.8 |
| Summit Z522 | 0.0 | 0.0 | 0.0 | 50.0 | 52.0 | 49.0 | 50.0 | 0.0 |
| (27.5%)* | | | | | | | | |
| Summit 498 | 57.0 | 57.0 | 63.0 | 0.0 | 0.0 | 0.0 | 0.0 | 0.0 |
| (28%)** | | | | | | | | |
| Westchlor | 0.0 | 0.0 | 0.0 | 0.0 | 0.0 | 0.0 | 0.0 | 50.0 |
| 100*** | | | | | | | | |
| Betaine | 1.0 | 2.0 | 0.0 | 0.0 | 0.0 | 0.0 | 1.0 | 0.0 |
| glycine | 0.0 | 0.0 | 0.0 | 0.0 | 0.0 | 0.0 | 0.0 | 1.30 |
| Water | 14.9 | 13.5 | 12.5 | 24.5 | 29.0 | 24.0 | 21.7 | 21.6 |
| DPG | 0.0 | 0.0 | 0.0 | 0.0 | 0.0 | 0.0 | 4 | 2.0 |
| MP diol | 0.0 | 0.0 | 0.0 | 0.0 | 0.0 | 2.45 | 0.0 | 0.0 |
| TPG | 2.0 | 2.0 | 2.0 | 0.0 | 0.0 | 0.0 | 0.0 | 0.0 |
| ZnCl2 sol (70.5 | 2.60 | 0.0 | 0.0 | 0.0 | 0.0 | 1.8 | 1.8 | 2.60 |
| w/w % aqu sol) | | | | | | | | |
| NaCl | 0.0 | 2.0 | 0.0 | 2.0 | 0.0 | 0.0 | 0.0 | 0.0 |
| ethanol | 2.5 | 3.5 | 2.5 | 2.5 | 0.0 | 2.50 | 2.50 | 2.5 |

*Al—Zr tetrachlorohydrex glycine complex (Z522, 27.5% in water from Summit Research Labs) which is a salt of the type described in Methods A and B above.
**Al—Zr tetrachlorohydrex glycine complex (Z498, 28% in water from Summit Research Labs).
***Aluminum dichlorohydrate complex (36% in water from Westwood Chemicals, Middletown NY)

TABLE B

| Ingredient | EX 9 | EX 10 |
|---|---|---|
| AZP-908* | 12.0 | 12.0 |
| MP Diol | 2.0 | 2.0 |
| Zinc Chloride | 3.0 | 3.0 |
| Propylene glycol | 1.0 | 1.0 |
| DI Water | 56.5 | 59.5 |
| Betaine | 5.0 | 2.0 |
| Cyclomethicone | 10.0 | 10.0 |
| PPG-3 | 2.0 | 2.0 |
| DC 1501 | 1.0 | 1.0 |
| DC 5225C Surfactant | 6.5 | 6.5 |
| Fragrance | 1.0 | 1.0 |

*Al—Zr tetrachlorohydrex glycine complex (Rezal AZP 908 powder from Reheis Berkeley Heights, NJ) to be dissolved in specified amount of water of the active phase.

1. The invention is a high viscosity, suspension-free white cream composition, free of elastomer, soap and borate gelling agents as well as any surfactants having an HLB>9, comprising:
(I) an oil phase which comprises:
(a) 7.0-28.4 weight % of one or more cyclomethicones having a flash point of 100 degrees C. or less;
(b) 0.6-2.0 weight % on an active basis of a silicone surfactant having a hydrophilic lipophilic balance <8;
(c) 0.0-3.0 weight % of a non-siliconized organic fragrance solubilizer comprising at least one silicone compatible ingredient selected from the group consisting of straight chain or branched hydrocarbons with a molecular weight less than 1000, alkyl substituted phenyl esters with an alkyl carbon chain length between C-1 to C-20, ethoxylated and/or propoxylated ethers with a carbon chain length from C-1 to C-25 and ethoxylation and/or propoxylation from 1-10, C12-15 alkyl benzoate, and PPG-3 myristyl ether;
(d) 1-5 weight % of a silicone emollient;
(e) 0-5 weight % fragrance or odor masking component; and
(II) an aqueous phase which comprises:
(a) 0.1-30 weight % on an anhydrous basis of a glycine containing antiperspirant active salt comprising either aluminum or aluminum and zirconium metals provided that (i) if an aluminum and zirconium salt is used then the metal/Cl ratio of the salt is in the range of 0.9-1.5:1, and the glycine/Zr ratio is >1; and (ii) if an aluminum salt is used, the aluminum to chloride molar ratio is in the range of 0.5-2.5:1, and the glycine/Al molar ratio is in the range of 0.05-0.26:1
wherein the glycine containing antiperspirant active salt has a pH in the range of 2-4 when measured in water at a concentration of 15% and is free of any other halide scavenging material;
(b) 30-70 weight % water;
(c) 0-3.0 weight % of a water soluble glycol system which comprises propylene glycol and an additional glycol component selected from the group consisting of ethylene glycol; diethylene glycol; triethylene glycol; tetraethylene glycol; propylene glycol; dipropylene glycol; tripropylene glycol; 1,3 propanediol; 2-methyl propanediol; methyl propanediol; 1,6-hexanediol; 1,3 butanediol; 1,4 butanediol; PEG-4 through PEG-600; PPG-9 through PPG-34; neopentyl glycol; trimethylpropanediol; 2,2 dimethyl-1,3propandiol; 2,2,4,4-tetramethyl-1,3-cyclobutane-diol; and mixtures thereof;
(d) 0.2-4 weight % of a monovalent or divalent ionizable, water soluble inorganic or organic salt of formula MₐX_{b} where a=1, or 2 and b=1 or 2; M is a member selected from the group consisting of Na⁺¹, Li⁺¹, K⁺¹, Mg⁺², Ca⁺², Sr⁺² and Zn⁺² and X is a member selected from the group consisting of chloride, bromide, iodide, citrate, gluconate, lactate, glycinate, glutamate, ascorbate, aspartate, nitrate, phosphate, hydrogenphosphate, dihydrogenphosphate, formate, maloneate, maleate, succinate, carbonate, bicarbonate, sulfate, hydrogensulfate;
(e) 0-5% of a water soluble carbon based emollient other than glycols;
(f) 0-10 weight % of an alcohol having 2-4 carbons; and
(g) 0-10% trimethylglycine;
wherein (i) all amounts are based on the weight of the entire composition, (ii) the cream composition is an emulsion having a viscosity in the range of 150,000-600,000 centipoise, and (iii) the composition has a ratio of oil phase to water phase in the range of 10:90 to 30:70.

2. A suspension-free white cream according to claim 1 having a viscosity in the range of 200,000 and 350,000 cps.

3. A suspension-free white cream according to claim 1 wherein the aluminum to chloride molar ratio is in the range of 0.05-0.26:1.

4. A suspension-free white cream according to claim 1 wherein the aluminum to chloride molar ratio is in the range of 0.05-0.16:1.

5. A suspension-free white cream according to claim 1 comprising 17-30 weight % of the antiperspirant active.

6. A suspension-free white cream according to claim 1 wherein the ionizable salt is selected from the group consisting of NaCl and ZnCl₂.

7. A suspension-free white cream according to claim 1 wherein the water soluble glycol system is a member selected from the group consisting of propylene glycol; dipropylene glycol; tripropylene glycol; 2-methyl-1,3 propanediol; 1,3 propanediol; methyl propylene glycol; low molecular weight polyethylene glycol; and mixtures of any of the foregoing.

8. A suspension-free white cream according to claim 1 wherein the water phase includes 1-5 weight % of ethanol.

9. A suspension-free white cream according to claim 1 additionally comprising 0.5-3 weight % of glycerin.

10. A suspension-free white cream according to claim 1 additionally comprising 0.5-3 weight % of trimethylglycine.

11. A suspension-free white cream according to claim 1 wherein the silicone surfactant is a silicone copolyol selected from the group consisting of compounds of Formulae I and II wherein:
(a) Formula I is:
(R¹⁰)₃—SiO—[(R¹¹)₂—SiO]ₓ—[Si(R¹²)(R^{b}—O—(C₂H₄O)ₚ—(C₃H₆O)ₛ—R^{c})O]_{y}—Si—(R¹³)₃ Formula I
wherein each of R¹⁰, R¹¹, R¹² and R¹³ may be the same or different and each is selected from the group consisting of C1-C6 alkyl; R^{b} is the radical —CₘH₂ₘ—; R^{c} is a terminating radical which is hydrogen, an alkyl group of one to six carbon atoms, an acyl group which is an ester having a terminating alkyl group of 1-4 carbons, or an aryl group which is phenyl; m has a value of two to eight; p and s have values such that oxyalkylene segment —(C₂H₄O)ₚ—(C₃H₆O)ₛ— has a molecular weight in the range of 200 to 5,000; the oxyalkylene segment has fifty to one hundred mole percent of oxyethylene units —(C₂H₄O)ₚ— and one to fifty mole percent of oxypropylene units —(C₃H₆O)ₛ—; x has a value of 8 to 400; and y has a value of 2 to 40;
(b) Formula II is:
(R¹⁰)₃—SiO—[(R¹¹)₂—SiO]ₓ—[Si(R¹²)(R^{b}—O—(C₂H₄O)ₚ—R^{c})O]_{y}—Si—(R¹³)₃ Formula II
wherein p has a value of 6 to 16; x has a value of 6 to 100; and y has a value of 1 to 20 and the other moieties have the same definition as defined in Formula I; and
(c) alternate embodiments of both Formulae I and II wherein the linking group R^{b}, the oxyalkylene segments, and the terminating radical R^{c} occupy positions bonded to the ends of the siloxane chain and are not bonded to a silicon atom in the siloxane chain.

12. A suspension-free white cream according to claim 1 wherein the fragrance solubilizer is selected from the group consisting of PPG-3 myristyl ether, hydrogenated polyisobutene, and C12-15 alkyl benzoate.

13. A suspension-free white cream according to claim 1 comprising 0.5-2 weight % of the fragrance solubilizer.

14. A suspension-free white cream according to claim 1 additionally comprising a fragrance or odor masking component.

15. A suspension-free white cream according to any one of claims 1-14 which is free of waxes, soap gelling agents, cellulosics and alginates.

16. A suspension-free white cream according to claim 1 comprising:
(a) 17-23 weight % of an oil phase comprising:
(i) 9-20 weight % of the cyclomethicones;
(ii) 0.6-1.0 weight % on an active basis of the silicone surfactant;
(iii) 1-2.5 weight % of the fragrance solubilizer; and
(iv) 1-2.5 weight % of a silicone emollient; and
(b) 77-83 weight % of an aqueous phase comprising:
(i) 0.1-30 weight % on an anhydrous basis of the antiperspirant active salt;
(ii) 1-3% weight % of the water soluble glycol system selected from the group consisting of propylene glycol, dipropylene glycol, tripropylene glycol, 2-methyl 1,3 propanediol, 1,3 propanediol, methyl propylene glycol, low molecular weight polyethylene glycol;
(iii) 1-4 weight % ethanol or propanol;
(iv) 0.8-3 weight % of the monovalent or divalent ionizable, water soluble inorganic or organic salt;
wherein the amounts are based on the weight of the entire composition.

17. A suspension-free white cream according to claim 1 comprising 0-3 weight % of propylene glycol , 2-methyl propane diol or dipropylene glycol or tripropylene glycol and combinations thereof.
